(19) United States
(12) Patent Application Publication
(10) Pub. No.: US 2008/0261955 A1
(43) Pub. Date: Oct. 23, 2008

(54) Use of Pharmaceutical Compositions of Lofepramine for the Treatment of Adhd, Cfs, Fm and Depression

(76) Inventors: Timothy Dinan, Cork (IE); Peter Daly, Co. Dublin (IE)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 11/576,325

(22) PCT Filed: Sep. 30, 2005
(86) PCT No.: PCT/IB05/02925

§ 371 Date: Mar. 18, 2008

Related U.S. Application Data
(60) Provisional application No. 60/614,541, filed on Oct. 1, 2004.
(60) Provisional application No. 60/637,728, filed on Dec. 22, 2004.

(51) Int. Cl. A61K 31/55; A61P 25/28; A61P 25/24

(52) U.S. Cl. 514/217

(57) ABSTRACT

In accordance with the present invention, it has been discovered that compounds exhibiting activity as a potent noradrenaline reuptake inhibitor (e.g., a NA:5HT ratio of greater than or equal to about 1000:1), and activity at the dopamine D2 receptor sites (e.g., lofepramine) are effective in the treatment and prevention of various diseases and disorders associated with noradreanaline reuptake, such as ADHD, CFS, fibromyalgia, and depression with pain.

### CROSS-REFERENCES TO RELATED APPLICATIONS

This application claims the benefit of the filing date of U.S. Provisional Application No. 60/614,541, filed Oct. 1, 2004 and U.S. Provisional Application No. 60/637,728, filed Dec. 22, 2004, each of which is herein incorporated by reference in its entirety.

### BACKGROUND OF THE INVENTION

Chronic Fatigue Syndrome (“CFS”) is a chronic, debilitating condition characterized by fatigue, cognitive problems, and various pains and other symptoms. The lives of patients are often frequently disrupted and as yet there is no effective therapy for this condition.

There are two criteria generally used for diagnosis of CFS. The criteria outlined by the US Centers for Disease Control and Prevention include medically unexplained fatigue of at least 6 months duration that is of new onset, not a result of ongoing exertion and not alleviated by rest. In addition, the diagnosis involves the determination of at least four additional symptoms including; tender lymph nodes, memory impairment, muscle pain, joint pain, headaches, un-refreshing sleep and post-exertional malaise. The Oxford criteria include severe disabling fatigue of at least 6 months duration that affects both physical and mental functioning with the fatigue being present for more than 50% of the time and also other symptoms including myalgia and sleep disturbances.

Fibromyalgia is a condition related to CFS. It is a common clinical condition presenting with musculoskeletal pain and tenderness often accompanied by fatigue (Goldberg D L, Curr Opin Rheumatol 7, 1995, 127-135). It is seen in both primary care and Rheumatology clinics, and is estimated to affect between 2-4% of the population. There were an estimated 14 million patients in the top seven pharmaceutical markets in 2002 (Decision Resources, Inc.). No specific treatment for the condition is yet available and it is frequently regarded as a functional disorder which can run a chronic course. The syndrome involves the presence of pain for over three months duration in all four quadrants of the body and additionally along the spine, as defined by the criteria of the American College of Rheumatology. Pain is found in at least 11 out of 18 points on the body. Also associated with the condition are non-restorative sleep, memory problems and fatigue.

Chronic Fatigue Syndrome and Fibromyalgia share many clinical characteristics. A majority of the patients are women and usually in their thirties or forties on initial presentation. Over 80% of both diagnostic categories complain of fatigue, myalgia, arthralgia, recurrent headaches and sleep difficulties (Moldofsky, 1993, Ciba Symposium 173, p 262-279).

A number of Selective Serotonin Reuptake Inhibitors {“SSRIs”) antidepressants have been tested for their utility in treatment of CFS, but have not been found effective. Also, the use of certain tricyclic antidepressants (“TCAs”) in the treatment of chronic pain associated with diabetic neuropathy and fibromyalgia has been investigated. TCAs provide moderate efficacy in such pain syndromes in about 50% of patients suffering from neuropathic pain, although their use is limited by their side effect profile. It has also been shown previously that certain TCAs in combination with an amino acid such as phenylalanine are useful in the treatment of pain and MS related fatigue (see, e.g., U.S. Pat. No. 6,441,038 and US 2002/0187958A1).

Further, the use of the antidepressant drug milnacipran to treat fibromyalgia and chronic fatigue syndrome has been investigated (see, e.g. U.S. Pat. No. 6,602,911 and U.S. Pat. No. 6,635,675). More particularly, milnacipran is described as being a dual serotonin norepinephrine reuptake inhibitor (“SNRI”) compound characterized by a non-tricyclic structure that is capable of inhibiting the reuptake of norepinephrine to an equal or greater extent than its inhibition of the reuptake of serotonin (referred to as a “NE >5-HT SNRI compound”, also milnacipran is also sometimes referred to as “NSRI” to indicate that its noradrenaline uptake inhibition is more potent than its serotonin reuptake inhibition). Such NE >5-HT SNRI compounds specifically disclaim TCAs. The potential utility of the novel antidepressant reboxetine which is a highly selective noradrenaline re-uptake inhibitor in fibromyalgia has also been claimed but without any supporting human data (U.S. Pat. No. 6,610,690). Reboxetine has not received approval for marketing within the United States at this time for any indication.

Attention deficit hyperactivity disorder (ADHD) has become recognised in recent years as a serious and chronic behavioural disorder of childhood. The condition involves two components, inattention and hyperactivity and it may manifest in either of these being predominant or in an equal combination.

Currently medicines that effectively treat ADHD target the dopamine and noradrenaline systems that are believed to be involved in the etiology of ADHD. In recent years many children with this condition have been treated by use of methylphenidate (“Ritalin”). However there are concerns over the possible addiction potential of this and similar stimulant drugs.

More particularly, there are certain requirements for effective therapies of ADHD in children. Such therapies must of course be safe and well tolerated. Previous use of tricyclic antidepressants such as desipramine demonstrated efficacy in limited trials and in off-label usage but toxicity was a significant factor. The effects of tricyclics on the acetylcholine receptors is of particular concern. Preferably such medications will have no abuse potential, unlike the case with the stimulant medications such as methylphenidate, and, preferably, such drugs will be long acting. Drugs which remove the need for children to receive additional doses during the day in school will be very helpful to the patient and caregivers.

Dysregulation of the central noradrenergic and dopinergic systems have been implicated in the patho-physiology of ADHD. The source of most of the noradrenaline in the CNS is the locus ceruleus, which has been shown to induce a waking alert state and to enhance information processing and attention to environmental stimuli.

Up until recently the only effective treatment has been use of “stimulant” medications which are believed to increase dopaminergic and noradrenergic transmission. These drugs, such as methylphenidate, are chemically related to amphetamines. Despite the advent of methylphenidate treatment there is still unmet medical need in this area. About 70% of patients who take a stimulant medication will see improvement in their core symptoms. A significant number of patients do not respond. In addition drugs in this class have a range of side effects such as weight loss, headaches, initial insomnia and in some cases irritability. Stimulants may also raise blood pressure and may exacerbate tics.

As mentioned above, tricyclic antidepressants have been used effectively in the treatment of ADHD. Drugs, such as desipramine and imipramine, have been studied in ADHD because they inhibit noradenraline re-uptake. The TCA's with greatest efficacy, which is comparable to the stimulants, are imipramine and desipramine. Both open and controlled trials have shown that imipramine at doses as low as 75 mg/day may begin to reduce hyperactivity in 3-10 days. In 1989, Biederman reported on a trial of desipramine in ADD (as defined in DSM III). The trial design was 62 children and adolescents randomly assigned to either desipramine or placebo. It was found that around 70% of the 31 patients treated with 4.6 mg/kg desipramine had a significant response. Sixty-nine percent of the study participants had previously failed to respond to or poorly tolerated stimulants.

Despite the foregoing, the use of TCA's in treating ADHD has declined greatly. Such drugs have a narrow therapeutic index and are cardiotoxic in overdose. Effective desipramine doses are quite high (4-5 mg/kg, which is equivalent to 290-350 mg/70 kg adult). Desipramine treatment is associated with asymptomatic and generally small increases in diastolic blood pressure, hear rate, and ECG conductance.

Recently, a new noradrenaline reuptake inhibitor, atomoxetine has been introduced for use in ADHD (“Strattera”). This drug provides clinical benefits and has a safe cardiovascular profile. The drug is primarily a noradrenaline reuptake inhibitor although it also has some effect on serotonin reuptake.

However, examination of two, large well-controlled studies have shown mean reduction of CAARS scores of 28.3% for atomoxetine vs. 18.1% for placebo. In the second study, these improvements were 30.1% vs. 19.6%, respectively. Overall these two studies have indicated a placebo response of about 20% and difference between active and placebo of about 10%. Moreover, atomoxetine is not a stimulant, a point that provides an effective marketing tool for this drug.

The drug milnacipran has been claimed as the first of a new class of “NSRI” drugs. These are drugs in which the reuptake activity for noradrenaline is more potent than for serotonin and which are not tricyclic antidepressants (see U.S. Pat. No. 6,602,911). Milnacipran is further claimed in International Publication WO 03068211 as a drug that has NSRI-NMDA properties in the use of ADHD and associated conditions. The latter patent application refers to various ways in which the relative potency of drugs may be compared and describes comparing IC 50 values and also relative Ki (inhibitor constants).

Both of these patents refer to the NA:5HT ratio for milnacipran as being 2:1 based on comparative IC 50 data International Publication WO 03068211 states that NSRI-NMDA compounds generally have a range of about 1.1-100:1 of NA:5HT. Compounds having a NA:5HT ratio in the range of about 2:1 to 10:1 are stated to be particularly effective.

Noradrenaline reuptake inhibitors have also been used in the treatment of ADHD and other diseases. Thus, International Publication WO 9952531 claims the use of reboxetine in ADHD (but no general claims directed to the mechanism of action).

Despite these studies, there remains a need for improved methods of treatment of CFA, fibromyalgia, depression with pain, and ADHD.

### BRIEF SUMMARY OF THE INVENTION

In accordance with the present invention, it has been found that lofepramine exhibits a unique combination of pharmacological actions, which, surprisingly, has been found to be useful in the treatment and prevention of a variety of diseases and disorders associated with noradrenaline reuptake.

In a first aspect of the invention, it has been unexpectedly found that lofepramine provides a highly effective and well tolerated treatment for ADHD. It is effective in both the treatment of behavioral aspects of ADHD, cognitive aspects of ADHD, behavioral and cognitive aspects of ADHD. In accordance with the present invention, lofepramine is effective in treating adult ADHD, pediatric ADHD, and adolescent ADHD.

As such, in certain aspects, the present invention generally relates to methods and compositions for treating ADHD. The methods and compositions of a preferred embodiment of the invention involve the use of lofepramine.

In other aspects of the present invention, it has been unexpectedly found that lofepramine, when administered alone in the absence of, e.g. any neurotransmitter precursor compounds or amino acids, provides a highly effective and well tolerated treatment for chronic fatigue syndrome (“CFS”). It is effective in both the treatment of affective/cognitive aspects of CFS, and also in the treatment of CFS associated pain and tenderness.

As such, the present invention generally relates to methods and compositions for treating CFS, fibromyalgia, or depression with pain. The methods and compositions of the invention involve the use of lofepramine.

These and other aspects will become apparent to those of skill in the art upon reading the present disclosure.

### DETAILED DESCRIPTION OF THE INVENTION

In accordance with the present invention, it has been found that lofepramine exhibits a unique combination of pharmacological actions, which, surprisingly, has been found to be useful in the treatment and prevention of a variety of diseases and disorders associated with noradrenaline reuptake. Without being bound by any particular mechanism of action, the main pharmacological actions relevant to the uses described herein include: (a) activity as a potent noradrenaline reuptake inhibitor (e.g., a NA:5HT ratio of greater than or equal to about 1000:1); and (b) activity at the dopamine D2 receptor sites. As such, in accordance with the present invention, compositions including compounds exhibiting such activity, such as lofepramine, are envisioned as being within the scope of the present invention. In a particularly preferred embodiment of the present invention, such a drug is lofepramine.

The selectivity a drug for various receptors may be compared by use of comparative Ki's and not comparative IC 50 values. (Bolden-Watson, C and Richelson, E., Life Sci, (52), 1993, 1023-1029), which is hereby incorporated by reference. For lofepramine the relative potencies of NA:5HT based on Ki data are 1, 200:1. As such, lofepramine is a NSRI with a very high NA:5HT ratio.

Significantly, the use of lofepramine, and compounds with the pharmacological profile described herein, provides a suitable manner to overcome safety concerns associated with use of highly effective but toxic TCAs known in the art, such as desipramine. In accordance with the present invention, it has been unexpectedly found that lofepramine has a very low cardiotoxicity, although its main metabolite is in fact desipramine. An analysis of fatal poisoning by antidepressants in Scotland, England and Wales was carried out by Buckley and Mcmanus (2002). Their data showed that for the tricyclic antidepressants as a class, fatalities per million prescriptions was 34.8; for desipramine it was 200.9; for the leading antidepressant venlafaxine it was 13.2 and for the SSRI class it was only 1.6. SSRI's are regarded by physicians as very safe drugs (which is their main advantage over TCA's). However, the data for lofepramine shows a fatality rate from overdose of 1.3 per million, prescriptions indicating that lofepramine is safer than the average for the SSRI class.

Arrythmia is the most serious consequence of TCA overdose. Progression of ECG changes are relatively predictable and related to the severity of the overdose. Mild oversose produces sinus tachycardia, mostly as a result of anticholinergic effects. More severe overdoses result in prolonger QRS and QTc intervals, followed by a prolonged PR interval and finally ventricular arrhythmias. Sjogren (1987) has demonstrated that tricyclic antidepressants, such as amitriptyline, imipramine and desipramine, prolong the ECG interval in rats infused with these antidepressants. In accordance with certain aspects of the present invention, lofepramine, on the other hand does not, and its effect is similar to that of the control vehicle.

In this regard, lofepramine is similar to atomoxetine and, accordingly, is very suitable for treating and ameliorating various conditions and disorders, e.g. including but not limited to those which TCAs are known to be useful. This safety aspect is particularly valuable in relation to possible accidental overdose in children or those subjects at risk of adverse cardiac events.

In accordance with certain aspects of the present invention, the dopaminergic activity of lofepramine as a valuable additional property in the treatment of a variety of conditions and disorders, e.g., ADHD, has been unexpected identified. Lofepramine has been shown to have potent activity at the D2 receptor. It is believed that the stimulant medications useful in treating ADHD symptoms act through both noradrenergic and dopaminergic mechanisms. Lofepramine is unique in that it possesses dual action functions on both norandrenergic and dopaminergic systems, but without belonging to the stimulant class.

As such, it has been unexpectedly found that lofepramine, optionally administered alone in the absence of any neurotransmitter precursor compounds, provides a highly effective and well tolerated treatment for ADHD. It is effective in both the treatment of behavioral aspects of ADHD, cognitive aspects of ADHD, behavioral and cognitive aspects of ADHD. In accordance with the present invention, lofepramine is effective in treating adult ADHD, pediatric ADHD, and adolescent ADHD.

As such, in certain aspects, the present invention generally relates to methods and compositions for treating ADHD. The methods and compositions of a preferred embodiment of the invention involve the use of lofepramine.

In other aspects of the present invention, it has been unexpectedly found that lofepramine, when administered alone in the absence of, e.g. any neurotransmitter precursor compounds or amino acids, provides a highly effective and well tolerated treatment for chronic fatigue syndrome (“CFS”). It is effective in both the treatment of affective/cognitive aspects of CFS, and also in the treatment of CFS associated pain and tenderness.

While not being bound by any one mechanisms of action, previous work by one of the co-inventors here, has supported the evidence that there is Hypothalamic-Pituitary-Adrenal axis under activity in CFS patients. (Scott L V, Medbak S and Dinan T G; Acta Psychiatr Scand 1998 June 97: 450-7). In this regard, it has been discovered that lofepramine induces upregulation of HPA activity along with symptomatic relief, which is believed to contribute to the effectiveness of the drug in the treatment of CFS.

As such, the present invention generally relates to methods and compositions for treating CFS, fibromyalgia, or depression with pain. The methods and compositions of the invention involve the use of lofepramine.

### A. Compounds of the Invention

The compounds of the present invention include lofepramine and its pharmaceutically acceptable salts, i.e., hydrochloride salt (the active ingredient in Gamanil™ and Lomont™). The chemical structure of lofepramine is shown below.

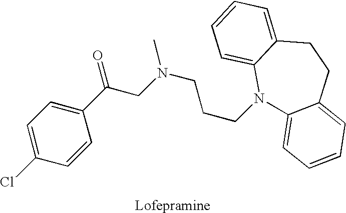

Lofepramine is a tricyclic antidepressant approved in a number of European countries including the UK and Ireland. Lofepramine is structurally similar to imipramine and is extensively metabolized to desipramine. It is believed that its antidepressant activity stems from the facilitation of noradrenergic neurotransmission by uptake inhibition, and possibly by the additional facilitation of serotoninergic neurotransmission. The overall therapeutic efficacy of lofepramine is comparable to that of imipramine, amitriptyline, clomipramine, maprotilene (maprotiline), and mianserin in patients with depression of varying severity and coexisting anxiety.

More particularly, lofepramine is a TCA that possesses a high NE (sometimes referred to as NA): 5-HT ratio and possesses stimulatory effects of 5-HT synthesis. Lofepramine also possesses dopaminergic effects and, very importantly, has very low cardiotoxicity. Additionally, it is noted that lofepramine possesses a NE:5-HT ratio that is higher than that of milnacipran.

As mentioned above, lofepmmine acts primarily as a NE reuptake inhibitor, although it also has 5-HT reuptake effects. In one study, lofepramine's NE IC50 was found to be 4 times that of its 5-HT IC50 (Segawa et al 1977). In a more accurate comparison of the relative potencies at NA and 5HT, Bolden-Watson showed that lofepramine has a NA over 5HT selectivity of 1,200:1. However, in accordance with the present invention, lofepramine has also been found to exert additional pharmacological properties. For instance, lofepramine has been shown to up-regulate serotonin synthesis in the brain. Lofepramine has also been shown to have a very low cardiotoxicity, with toxic levels similar to that found in the SSRI's. Further, lofepramine has been found to exert its effects on dopamine D2 receptors. Unlike a number of other tricyclic antidepressants lofepramine does not induce sedation. While any compound which possesses properties similar to lofepramine in these respects may be useful in the present invention, lofepramine and its pharmaceutically acceptable salts are the preferred compound of the invention

Also falling within the scope of the present invention are the in vivo metabolic products of the lofepramine compounds described herein, including desipramine. Such products may result, for example, from the oxidation, reduction, hydrolysis, amidation, esterification, and the like of the administered compound, primarily due to the enzymatic processes. Accordingly, the invention includes compounds produced by a process comprising contacting a lofepramine compound of the invention with a mammalian tissue or a mammal for a period of time sufficient to yield a metabolic product thereof. Such products typically are identified by preparing a radio-labeled (e.g. C¹⁴ or H³) lofepramine compound of the invention, administering it in a detectable dose (e.g., greater than about 0.5 mg/kg) to a mammal such as a rat, mouse, guinea pig, monkey, or human, allowing sufficient time for metabolism to occur (typically about 30 seconds to 30 hours), and isolating its conversion products from urine, blood, tumor, or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites may be done in the same way as conventional drug metabolism studies well-known to those skilled in the art. The conversion products, so long as they are not otherwise found in vivo, are useful in diagnostic assays for therapeutic dosing of the compounds of the invention, even if they possess no biological activity of their own.

The main metabolite of lofepramine is the tricyclic drug desipramine. Desipramine may contribute to the pharmacological actions of lofepramine but lofepramine is not a pro-drug for desipramine and lofepramine has a of NA:5HT ratio of about four times that of desipramine. However, the non-toxicity of lofepramine in overdose appears not be fully understood since it would be expected that in such cases significant plasma levels of desipramine would be generated.

The metabolites of lofepramine include three compounds that are also common to the metabolism of imipramine, namely, desipramine, 2-hydroxydesipraminne, and didesmethylimipramine. Lofepramine also generates three unique metabolites of which two have been identified as 2-hydroxyllofepramine and desmethyllofepramine (Strangarden, K and P. O. Gunnarsson. 1994. Metabolism of lofepramine and imipramine in liver microsomes from rat and man. Xenobiotica, 24, No. 8, 703-711), which is hereby incorporated by reference.

In another aspect of the invention, it is believed that the unique metabolites of lofepramine act in a cardioprotective manner to counter the toxic effects of the desipramine metabolite. If so, then these metabolites, depending on their pharmacokinetics, may also be safe and effective drugs for the treatment of the conditions and disorders described herein. In a highly preferred embodiment of the present invention, 2-hydroxyllofepramine and desmethyllofepramine are compounds of the present invention, either individually or in combination or in the ratios in which they occur following metabolism of lofepramine.

### B. Methods of the Invention

In accordance with the present invention, it has been discovered that compounds exhibiting activity as a potent noradrenaline reuptake inhibitor (e.g. a NA:5HT ratio of greater than or equal to about 1000:1), and activity at the dopamine D2 receptor sites (e.g., lofepramine) are effective in the treatment and prevention of various diseases and disorders associated with noradrenaline reuptake, such as ADHD, CFS, fibromyalgia, and depression with pain.

As such, one aspect of the invention relates to methods for treating either predominantly inattention type or hyperactivity-impulsive type ADHD in a subject in need thereof, comprising administering to the patient a composition comprising a therapeutically effective amount of lofepramine or a pharmaceutically acceptable salt thereof. Another aspect of the invention relates to methods for treating combined type ADHD in a subject in need thereof, comprising administering to the patient a composition comprising a therapeutically effective amount of lofepramine or a pharmaceutically acceptable salt thereof.

Another aspect of the invention relates to methods for treating chronic fatigue syndrome (“CFS”) in a subject in need thereof, comprising administering to the patient a composition comprising a therapeutically effective amount of lofepramine or a pharmaceutically acceptable salt thereof. In a preferred embodiment, the composition is free of amino acids such as phenylalanine. In another embodiment, lofepramine may be employed as the single active ingredient of a medicament for the treatment of CFS, i.e., the composition consists essentially of lofepramine or a pharmaceutically acceptable salt thereof.

Another aspect of the invention relates to methods for treating fibromyalgia in a subject in need thereof, comprising administering to the patient a composition comprising a therapeutically effective amount of lofepramine or a pharmaceutically acceptable salt thereof. In a preferred embodiment, the composition is free of amino acids such as phenylalanine. In another embodiment, lofepramine may be employed as the single active ingredient of a medicament for the treatment of fibromyalgia, i.e., the composition consists essentially of lofepramine or a pharmaceutically acceptable salt thereof.

While the use of lofepramine in the treatment of depression is generally known, it was unexpectedly discovered in accordance with the present invention that lofepramine is particularly effective in the treatment of depression with painful symptoms or “somatizations” of depression. Such conditions should not be confused with fibromyalgia, as the diagnosis of the latter excludes major depression. As such, in one embodiment of the invention, lofepramine may be employed as the single active ingredient of a medicament for the treatment of “depression with painful symptoms”, i.e., the composition consists essentially of lofepramine or a pharmaceutically acceptable salt thereof.

In another aspect of the invention, due to the unexpected discovery of the cardioprotective aspects of the compounds of the present invention, the methods of the invention may include identification of a subject at risk of an adverse cardiac event. As such, in another aspect of the invention, a method for treating or preventing depression in a subject in need thereof is provided, comprising administering to the patient a composition comprising a therapeutically effective amount of lofepramine or a pharmaceutically acceptable salt thereof, wherein the subject is at risk of an adverse cardiac event. In certain embodiments, the method comprises identifying the subject as being at risk of an adverse cardiac event. Adverse cardiac events include any cardiac event generally recognized by those skilled in the art, including myocardial infarction, congestive heart failure, irregular heat beat, stroke, etc.

In one embodiment of the present invention, lofepramine in immediate release form is administered to the subject, e.g., a pediatric subject, more than once a day. The first dose is in the morning, such that the dose and half-life of the drug are sufficient to provide effective treatment during school or work hours.

In one embodiment of the present invention, lofepramine in immediate release form, but at a higher dose than in the previous embodiment, is administered once a day in the morning. Given the safety of lofepramine, once a day dosing can be achieved for the purposes herein without recourse to any sustained release technologies.

In a preferred embodiment lofepramine may be administered in a extended release once a day format using techniques known in the art. The once a day form of lofepramine will provide additional benefits in further reducing the already mild side effects of the immediate release form and also have the convenience of once a day dosing.

According to the methods of the invention, the compound(s) may be administered to the subject via any drug delivery route known in the art. Specific exemplary administration routes include peripheral and central routes such as oral, ocular, rectal, buccal, topical, nasal, ophthalmic, subcutaneous, intramuscular, intraveneous (bolus and infusion), intracerebral, transdermal, and pulmonary. In a preferred embodiment, the composition is administered orally via tablet.

The term “therapeutically effective amount”, as used herein, refers to an amount of a pharmaceutical agent to treat, ameliorate, or prevent the identified disease or condition, or to exhibit a detectable therapeutic or inhibitory effect. The effect can be detected by any means known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

More particularly, preferred therapeutically effective amounts of the compound(s) of the present invention include administration at doses that vary from 40 mg to 420 mg, administered in single or divided doses, depending upon the route of administration and the age and size of the subject, as recognized by those skilled in the art. Guidance as to particular dosages and methods of delivery is provided in the literature and is generally available to practitioners in the art. Recommended dosages for lofepramine as employed in practice the present invention are 70 mg twice daily (140 mg), or up to three times per day (210 mg) depending on patient response. Lofepramine may also be employed up to doses of 420 mg per day given its low cardiotoxicity. Thus, in one embodiment, doses of lofepramine range from about 70 mg to about 140 mg per day, about 140 mg to about 210 mg per day, or about 210 mg to about 420 mg per day. Higher doses of lofepramine may be employed for shorter periods (such as one to two weeks) in order to obtain immediate or short term relief from painful symptoms.

The exact dosage will be determined by the practitioner, in light of factors related to the subject that requires treatment and the form of lofepramine used (e.g., the salt form). Dosage and administration are adjusted to provide sufficient levels of the active agent(s) or to maintain the desired effect. Other factors which may be taken into account include the severity of the disease state, general health of the subject, age and weight of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

### C. Pharmaceutical Compositions of the Invention

In yet another aspect of the present invention, pharmaceutical compositions useful in the methods of the invention are provided. The pharmaceutical compositions of the invention may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8.0.

In a particularly preferred embodiment of the present invention, the pharmaceutical compositions of the invention comprise a therapeutically effective amount of lofepramine or a pharmaceutically acceptable salt thereof, together with one or more pharmaceutically acceptable excipients. For instance, when the pharmaceutical composition is formulated as an oral tablet, the composition preferably comprises from about 0.1 mg to about 70 mg of the lofepramine compound, more preferably from about 5 mg to about 100 mg. As discussed above, the exact amount of lofepramine may vary. In another preferred embodiment, the pharmaceutical composition is entirely free from amino acids such as phenylalanine. In another preferred embodiment, the pharmaceutical composition comprises the lofepramine compound as its only active ingredient, i.e., there are no other active ingredients included in the pharmaceutical composition.

In a more particularly preferred embodiment of the present invention, the pharmaceutical compositions of the invention comprise a tablet, capsule, lozenge or other orally available drug which comprises a single dose of lofepramine or a pharmaceutically acceptable salt suitable to provide effective once a day therapy for the conditions herein.

In an alternative embodiment of the present invention, the pharmaceutical compositions of the invention may comprise a combination of active ingredients, including but not limited to a second therapeutic agent useful in the treatment of ADHD, CFS, fibromyalgia, and/or depression with pain. Therapeutic amounts of second agents are generally known in the art or may be determined by the skilled clinician.

Formulations of the present invention, e.g. for parenteral or oral administration, are most typically solids, liquid solutions, emulsions or suspensions, while inhaleable formulations for pulmonary administration are generally liquids or powders, with powder formulations being generally preferred.

The term “pharmaceutically acceptable excipient” refers to an excipient for administration of a pharmaceutical agent, such as the compounds of the present invention. The term refers to any pharmaceutical excipient that may be administered without undue toxicity. Pharmaceutically acceptable excipients are determined in part by the particular composition being administered, as well as by the particular method used to administer the composition. Accordingly, there exists a wide variety of suitable formulations of pharmaceutical compositions of the present invention (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions of the invention may be formulated in any form suitable for the intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed. Tablets can be formulated as controlled release drugs using techniques known in the art so as to provide once a day dosing within the ranges as specified herein.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions of the invention may be formulated as suspensions comprising a compound of the present invention in admixture with at least one pharmaceutically acceptable excipient suitable for the manufacture of a suspension. In yet another embodiment, pharmaceutical compositions of the invention may be formulated as dispersible powders and granules suitable for preparation of a suspension by the addition of suitable excipients.

Excipients suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring, or a coloring agent, or a combination of these.

### D. Combination Therapy

In another aspect of the invention, it has been unexpectedly discovered that lofepramine may be combined with another active ingredient to treat ADHD, CFS, fibromyalgia, and depression with pain. More particularly, SNRI and NSRI drugs which are single molecules cannot vary the ratio of NE:5-HT activity. However, in accordance with the invention, such variation has been found to be desirable, particularly in the treatment of depression with pain. As such, another aspect of the invention relates to the combination of a SNRI or NSRI (i.e., a primary NE reuptake inhibitor) with a primary 5-HT reuptake inhibitor. In a preferred embodiment, the NE:5-HT ratio employed is greater than 1:1, more preferably in the range about 2-10:1, and even more preferably between about 10-100:1.

Thus, in another embodiment of the invention, lofepramine, which is primarily a NE reuptake inhibitor, is combined with a compound which is primarily a 5-HT reuptake inhibitor. In a preferred embodiment, lofepramine is combined with citalopram. However, the skilled artisan will recognize that a variety of active ingredients may be administered in combination with the primary NE reuptake inhibitor that may act to augment or synergistically enhance the activity of the primary NE reuptake inhibitor (e.g. lofepramine). Therapeutic doses may be determined by one of ordinary skill in the art. In a particularly preferred embodiment of the invention, 5 mg of citalopram and 100 mg of lofepramine are administered daily.

The primary NE reuptake inhibitor (e.g., lofepramine) and the primary 5-HT reuptake inhibitor (e.g., citalopram) may be combined in any manner known in the art such as a unitary dosage form, or in separate dosage forms intended for simultaneous or sequential administration to a patient in need of treatment. When administered sequentially, the combination may be administered in two or more administrations. In an alternative embodiment, it is possible to administer one or more compounds of the present invention and one or more additional active ingredients by different routes.

According to the methods of the invention, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods of the invention may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

To assist in understanding the present invention, the following Examples are included. The experiments relating to this invention should not, of course, be construed as specifically limiting the invention and such variations of the invention, now known or later developed, which would be within the purview of one skilled in the art are considered to fall within the scope of the invention as described herein and hereinafter claimed.

### EXAMPLES

The present invention is described in more detail with reference to the following non-limiting examples, which are offered to more fully illustrate the invention, but are not to be construed as limiting the scope thereof. The examples illustrate the preparation of certain compounds of the invention, and the testing of these compounds. Those of skill in the art will understand that the techniques described in these examples represent techniques described by the inventors to function well in the practice of the invention, and as such constitute preferred modes for the practice thereof. However, it should be appreciated that those of skill in the art should in light of the present disclosure, appreciate that many changes can be made in the specific methods that are disclosed and still obtain a like or similar result without departing from the spirit and scope of the invention.

### Example 1

Patient is a 19 year old man with a diagnosis of DSM-IV ADHD. His education was severely impacted by an inability to sustain attention for any length of time and he was also very disruptive in class. His inability to sustain attention was causing difficulty at work. He was referred by his primary care physician. He was commenced on lofepramine 70 mg twice daily. After 1 week his parents noted a significant improvement in his general behaviour and his concentration in particular. The dose of lofepramine was increased to 70 mg three times daily. There was continued improvement, which was sustained for 3 months at which point he decided to stop the medication. Within 48 hours his concentration deteriorated and he was once again in trouble at work. A week later the medication was recommenced and he has now remained well for approximately 4 months. He is performing effectively at work and his parents are happy with the situation.

A 17 year old boy had been on methylphenidate for 2 years which was prescribed for ADHD characterised by poor concentration and impulsive behaviour. He stopped taking the medication and deteriorated. When he attended the clinic he was commenced on lofepramine 70 mg twice daily. No immediate improvement in behaviour was noted. The dose was increased to 70 mg three times daily and his parents reported a major improvement. This has been sustained for 4 months. He reports no side-effects.

### Example 2

A study was carried out to access the efficacy of lofepramine compared to placebo in 20 adult patients with ADHD at a single study centre. Active treatment was for 3 months. Patients with DSM-IV diagnosis of ADHD were recruited. Patients were assessed using the patient-rated Connors Adult ADH Rating Scales (CAARS). Initially, there was a single blind placebo period between day 0 and week 3, and patients who responded to placebo were withdrawn. Oral administration of lofepramine (140 mg/day) was commenced from week 3 onwards. Patients showing little response to this dose and tolerable side effects were titrated upwards to 210 mg/day and then to 280 mg/day. At the sixth visit at week 12, patients were gradually discontinued by 70 mg decrements over a 4 day period with all medication discontinued by week 14. The results show significant benefit over placebo as assessed by CAARS scores.

### Example 3

Forty-four patients meeting the criteria for Chronic Fatigue Syndrome were recruited in a cross-over design. Patients had at least a 6 month history of chronic fatigue syndrome, no co-morbid psychiatric disorder (HADS scale for entry <10), negative for drugs of abuse, normal ECG and had 4 or more of the following symptoms; substantial impairment of short term memory or concentration, sore throat, tender lymph nodes, muscle pain, multi-joint pain without swelling or redness, headaches of a new type, pattern or severity, unrefreshing sleep, post-exertional malaise lasting more than 24 hours. Patients were administered lofepramine at doses up to 210 mg/day over the 26 weeks of the study. Patients were assessed using the FibroFatigue Scale (interview scale) and the CDR battery of cognitive tests (computer based assessment of cognition). Other assessments included HADS and Clinical Global Improvement (CGI). Four CDR assessments and 9 Fibrofatigue assessments were carried out. Data showed that lofepramine produced a significant improvement on all scales.

1.-20. (canceled)

21. A method for the treatment or prevention of a disease or disorder associated with noradrenaline reuptake, said method comprising administering a pharmaceutical composition comprising a therapeutically or prophylactically effective amount of lofepramine or a pharmaceutically acceptable salt thereof to a subject in need thereof.

22. A method according to claim 21, wherein said pharmaceutical composition is essentially free of amino acids.

23. A method according to claim 21, wherein pharmaceutical composition comprises from about 40 mg to about 400 mg of said lofepramine or pharmaceutically acceptable salt thereof.

24. A method according to claim 21, wherein pharmaceutical composition comprises from about 70 mg to about 420 mg of said lofepramine or pharmaceutically acceptable salt thereof.

25. A method according to claim 21, wherein pharmaceutical composition comprises from about 70 mg to about 140 mg of said lofepramine or pharmaceutically acceptable salt thereof.

26. A method according to claim 21, wherein pharmaceutical composition comprises from about 140 mg to about 210 mg of said lofepramine or pharmaceutically acceptable salt thereof.

27. A method according to claim 21, wherein pharmaceutical composition comprises from about 210 mg to about 420 mg of said lofepramine or pharmaceutically acceptable salt thereof.

28. A method according to claim 21, wherein said subject is at risk of an adverse cardiac event.

29. A method according to claim 21, wherein said disease or disorder is selected from the group consisting of Attention Deficit Hyperactivity Disorder (ADHD), chronic fatigue syndrome (“CFS”), fibromyalgia, and depression with associated painful symptoms.

30. A method according to claim 29, wherein said disease or disorder is inattentive type of ADHD.

31. A method according to claim 29, wherein said disease or disorder is a hyperactive-impulsive type of ADHD.

32. A method according to claim 29, wherein said disease or disorder is a combined type of ADHD.

33. A method according to claim 29, wherein said disease or disorder is CFS.

34. A method according to claim 29, wherein said disease or disorder is fibromyalgia.

35. A method according to claim 29, wherein said disease or disorder is depression with associated painful symptoms.

36. A method according to claim 35, wherein pharmaceutical composition further comprises a second component which is primarily a 5-HT reuptake inhibitor.

37. A method according to claim 36, wherein said 5-HT reuptake inhibitor is citalopram.

38. A method according to claim 36, wherein the ratio of the lofepramine to second component is greater than 1:1.

39. A method for the treatment or prevention of a disease or disorder associated with noradrenaline reuptake, said method comprising administering a pharmaceutical composition comprising a therapeutically or prophylactically effective amount of a compound exhibiting a NA:5HT ratio f greater than or equal to about 1000:1 and activity at the dopamine D2 receptor sites to a subject in need thereof.

40. A method according to claim 39, wherein said compound is lofepramine or a pharmaceutically acceptable salt thereof.

41. A method according to claim 39, wherein said compound consists essentially of lofepramine or a pharmaceutically acceptable salt thereof.

42. A method according to claim 39, wherein said pharmaceutical composition is essentially free of amino acids.

43. A method according to claim 39, wherein said pharmaceutical composition comprises about 40 mg to about 400 mg of said compound.

44. A method according to claim 39, wherein said pharmaceutical composition comprises about 70 mg to about 420 mg of said compound.

45. A method according to claim 39, wherein said pharmaceutical composition comprises about 70 mg to about 140 mg of said compound.

46. A method according to claim 39, wherein said pharmaceutical composition comprises about 140 mg to about 210 mg of said compound.

47. A method according to claim 39, wherein said pharmaceutical composition comprises about 210 mg to about 420 mg of said compound.

48. A method according to claim 39, wherein said subject is at risk of an adverse cardiac event.

49. A method according to claim 39, wherein said disease or disorder is selected from the group consisting of Attention Deficit Hyperactivity Disorder (ADHD), chronic fatigue syndrome (“CFS”), fibromyalgia, and depression with associated painful symptoms.

50. A method according to claim 49, wherein said disease or disorder is inattentive type of ADHD.

51. A method according to claim 49, wherein said disease or disorder is a hyperactive-impulsive type of ADHD.

52. A method according to claim 49, wherein said disease or disorder is a combined type of ADHD.

53. A method according to claim 49, wherein said disease or disorder is CFS.

54. A method according to claim 49, wherein said disease or disorder is fibromyalgia.

55. A method according to claim 49, wherein said disease or disorder is depression with associated painful symptoms.

56. A method according to claim 55, wherein said pharmaceutical composition further comprises a second component which is primarily a 5-HT reuptake inhibitor.

57. A method according to claim 56, wherein the 5-HT reuptake inhibitor is citalopram.

58. A method according to claim 56, wherein the ratio of the said compound to second component is greater than 1:1.
